(19) United States
(12) Patent Application Publication
(10) Pub. No.: US 2025/0239741 A1
(43) Pub. Date: Jul. 24, 2025

(54) BATTERY MODULE AND ENERGY STORAGE SYSTEM INCLUDING THE SAME

(71) Applicant: Samsung SDI Co., Ltd., Yongin-si (KR)

(72) Inventor: Yusik Hwang, Yongin-si (KR)

(73) Assignee: Samsung SDI Co., Ltd., Yongin-si (KR)

(21) Appl. No.: 18/755,336

(22) Filed: Jun. 26, 2024

(30) Foreign Application Priority Data
Jan. 18, 2024 (KR) 10-2024-0008372

(51) Int. Cl. H01M 50/507; H01M 10/42; H01M 10/48; H01M 50/143

(52) U.S. Cl. CPC H01M 50/507; H01M 10/425; H01M 10/482; H01M 10/486; H01M 50/143; H01M 2010/4271

(57) ABSTRACT

Provided is a battery module and energy storage system including the same. The battery module includes a plurality of battery cells, a plurality of busbars electrically connecting the plurality of battery cells, a busbar holder positioned above the plurality of battery cells and supporting the plurality of busbars, a plurality of gas flow detection sensors fixedly coupled to the busbar holder, and circuitry electrically connected to the plurality of busbars and the plurality of gas flow detection sensors.

### CROSS-REFERENCE TO RELATED APPLICATION

The present application claims priority to and the benefit under 35 U.S.C. § 119(a)-(d) of Korean Application No. 10-2024-0008372, filed on Jan. 18, 2024, in the Korean Intellectual Property Office, the entire contents of which are incorporated by reference herein.

### FIELD

Aspects of the present disclosure relate to a battery module and energy storage system including the same.

### BACKGROUND

Unlike primary batteries that are not designed to be (re)charged, secondary (or rechargeable) batteries are batteries that are designed to be discharged and recharged. Low-capacity secondary batteries are used in portable, small electronic devices, such as smart phones, feature phones, notebook computers, digital cameras, and camcorders, while large-capacity secondary batteries are widely used as power sources for driving motors in hybrid vehicles and electric vehicles and for storing power (e.g., home and/or utility scale power storage). A secondary battery generally includes an electrode assembly composed of a positive electrode and a negative electrode, a case accommodating the same, and electrode terminals connected to the electrode assembly.

The above information disclosed in this Background section is for enhancement of understanding of the background of the present disclosure, and therefore, it may contain information that does not constitute related (or prior) art.

### SUMMARY

Embodiments of the present disclosure provide a battery module and energy storage system including the same.

These and other aspects and features of the present disclosure will be described in or will be apparent from the following description of embodiments of the present disclosure.

A battery module according to one or more embodiments of the present disclosure may include: a plurality of battery cells; a plurality of busbars electrically connecting the plurality of battery cells; a busbar holder positioned above the plurality of battery cells and supporting the plurality of busbars; a plurality of gas flow detection sensors fixedly coupled to the busbar holder; and circuitry electrically connected to the plurality of busbars and the plurality of gas flow detection sensors.

According to one or more embodiments of the present disclosure, the plurality of gas flow detection sensors may include: sensor bodies and a plurality of first engagement portions.

According to one or more embodiments of the present disclosure, the busbar holder may include: a plurality of second engagement portions provided to respectively correspond to the plurality of first engagement portions, and the plurality of second engagement portions is positioned at a bottom surface of the busbar holder to face the plurality of battery cells.

According to one or more embodiments of the present disclosure, the plurality of gas flow detection sensors is fixedly coupled to the bottom surface of the busbar holder by respectively engaging the plurality of first engagement portions with the plurality of second engagement portions.

According to one or more embodiments of the present disclosure, a first one of the plurality of first engagement portions is formed in a protruding hook shape, a first one of the plurality of second engagement portions is formed in a groove shape for receiving the corresponding one of the plurality of the first engagement portions, and the first one of the plurality of first engagement portions is engaged with the first one of the plurality of second engagement portions by inserting the first one of the plurality of first engagement portions into the first one of the plurality of second engagement portions.

According to one or more embodiments of the present disclosure, vents are respectively formed at top surfaces of the plurality of battery cells, and the plurality of gas flow detection sensors is respectively disposed above the vents of the plurality of battery cells.

According to one or more embodiments of the present disclosure, each of the plurality of gas flow detection sensors detects at least one of a wind pressure, a wind speed, or an air volume of vent gas discharging from the corresponding vent.

According to one or more embodiments of the present disclosure, an energy storage system (ESS) may include: the battery module; and a battery rack in which the battery module is accommodated.

According to one or more embodiments of the present disclosure, the energy storage system (ESS) may further include: a fire extinguishing system; and a battery management system (BMS) electrically connected to the battery module, wherein the circuitry of the battery module comprises a battery management module (BMM), each of the plurality of gas flow detection sensors transmits, in response to detecting a flow of gas, a gas flow detection signal to the BMM, and the BMM transmits, in response to receiving the gas flow detection signal, an extinguishing preparation signal to the BMS.

According to one or more embodiments of the present disclosure, the fire extinguishing system may include: an agent container storing an extinguishing agent; a main valve configured to open and close the agent container; a main pipe through which the extinguishing agent is transferred from the agent container; branch pipes branching from the main pipe to supply the extinguishing agent to each of the plurality of battery cells; and a plurality of extinguishing nozzles provided along each of the branch pipes at positions corresponding to positions of the plurality of battery cells, respectively, wherein, in response to receiving the extinguishing preparation signal, the BMS controls the main valve to be opened.

According to one or more embodiments of the present disclosure, each of the plurality of extinguishing nozzles may include: a heat-sensitive member that is configured to melt in response to a temperature of a corresponding battery cell of the plurality of battery cells being equal to or greater than a predetermined threshold, the melting of the heat-sensitive member being configured to cause the extinguishing agent to be sprayed onto the corresponding battery cell of the plurality of battery cells.

According to one or more embodiments of the present disclosure, each of the branch pipes is formed by injection holes disposed at positions respectively corresponding to battery cells of the plurality of battery cells, and the heat-sensitive member is disposed at a position at which the heat-sensitive member blocks one of the injection holes.

According to one or more embodiments of the present disclosure, vents are respectively formed at top surfaces of each of the plurality of battery cells, and the plurality of gas flow detection sensors are respectively disposed above the vents, wherein the fire extinguishing system may further include: a fire detection sensor; and a plurality of extinguishing valves that open and close the plurality of extinguishing nozzles, respectively, wherein the fire detection sensor transmits, in response to detecting a fire, a fire detection signal to the BMS, and the BMS controls, in response to receiving the fire detection signal, a particular extinguishing valve to be opened among the plurality of extinguishing valves, the particular extinguishing valve being associated with a particular battery cell among the plurality of battery cells for which the flow of gas has been detected.

According to one or more embodiments of the present disclosure, the extinguishing agent is transferred from the agent container to the main pipe and the branch pipes after the BMS receives the extinguishing preparation signal and before the BMS receives the fire detection signal.

According to one or more embodiments of the present disclosure, upon opening the particular extinguishing valve, the extinguishing agent is immediately sprayed into the particular battery cell through a particular extinguishing nozzle associated with the particular extinguishing valve among the plurality of extinguishing nozzles.

According to one or more embodiments of the present disclosure, the fire detection sensor comprises a smoke detection sensor.

According to one or more embodiments of the present disclosure, in response to receiving at least one of the extinguishing preparation signal or the fire detection signal, the BMS transmits an alert signal to an external device.

According to one or more embodiments of the present disclosure, the fire extinguishing system may further include: a gas concentration detection sensor; and an active venting unit, wherein, in response to detecting, by the gas concentration detection sensor, a gas concentration that is equal to or greater than a predetermined threshold, the gas concentration detection sensor is configured to transmit a gas detection signal to the BMS, and the BMS is configure to activate the active venting unit in response to receiving the gas detection signal.

According to one or more embodiments of the present disclosure, the fire extinguishing system may further include: a gas concentration detection sensor; and a door that opens and closes a first side of the energy storage system, wherein, in response to detecting, by the gas concentration detection sensor, a gas concentration that is equal to or greater than a predetermined threshold, the gas concentration detection sensor is configured to transmit a gas detection signal to the BMS, and the BMS is configured to control the door to cause the door to be opened in response to receiving the gas detection signal.

According to one or more embodiments of the present disclosure, in response to the door being opened, the BMS transmits a door opening notification to an external device.

According to embodiments of the present disclosure, individual monitoring of each of the battery cells can be carried out using the plurality of gas flow detection sensors. Consequently, specific battery cells exhibiting abnormal behaviors such as vent gas discharge can be detected at an early stage. Thus, an accurate and rapid response to the abnormal behaviors in specific battery cells can be achieved.

According to embodiments of the present disclosure, transfer of the extinguishing agent is initiated before smoke is detected by the fire detection sensor, thereby reducing the response time to fire.

According to embodiments of the present disclosure, fire response scenarios corresponding to abnormalities detected by the gas flow detection sensor, the fire detection sensor, and the gas concentration detection sensor can be executed in parallel simultaneously. With this configuration, the battery management system (BMS) can quickly and accurately detect and identify abnormally behaving battery cells, thereby facilitating early fire suppression and minimizing fire propagation.

According to embodiments of the present disclosure, the extinguishing agent can be transferred to reach all of the battery cells included in the energy storage system. Further, by connecting the branch pipes to the upper region of the respective battery modules, the extinguishing agent can be supplied to all of the battery cells even when the plurality of battery modules are aligned adjacent to each other in a front-to-back direction, a left and right direction, and/or a up and down direction. Accordingly, the extinguishing agent can be sprayed intensively onto battery cells exhibiting abnormal behavior (for example, vent gas discharge, fire outbreak, and the like).

According to embodiments of the present disclosure, the main valve of the agent container (fire extinguishing vessel) can be opened in advance by detecting the flow of vent gas before a fire occurs in the battery cell. Further, in the event of the fire in the battery cell, the fire can be prevented from spreading to neighboring battery cells or neighboring battery modules by delivering the fire extinguishing agent directly to the battery cell. Furthermore, even if the fire spreads to the neighboring battery cells, the extinguishing valves can be opened to immediately supply the extinguishing agent when the gas flow detection sensors associated with the neighboring battery cells detect the flow of gas. This minimizes the damage caused by the spread of the fire.

However, aspects and features of the present disclosure are not limited to those described above, and other aspects and features not mentioned will be clearly understood by a person skilled in the art from the detailed description, described below.

### BRIEF DESCRIPTION OF DRAWINGS

The following drawings attached to this specification illustrate embodiments of the present disclosure, and further describe aspects and features of the present disclosure together with the detailed description of the present disclosure. Thus, the present disclosure should not be construed as being limited to the drawings:

FIG. 1 illustrates a perspective view showing an example of a battery cell 100 according to embodiments of the present disclosure.

FIG. 2 is an exploded perspective view showing an example in which a part of a battery module is disassembled according to one embodiment of the present disclosure.

FIG. 3 is a cross-sectional view illustrating a gas flow detection sensor coupled to a busbar holder according to one embodiment of the present disclosure.

FIG. 4 is a diagram illustrating an example in which a gas flow detection sensor is fastened to a busbar holder according to one embodiment of the present disclosure.

FIG. 5 is a diagram illustrating an example of a fire extinguishing system included in an energy storage system according to one embodiment of the present disclosure.

FIG. 6 is a block diagram illustrating an operational flow of a fire extinguishing system according to one embodiment of the present disclosure.

FIG. 7 is a diagram illustrating an installation location A of a branch pipe within an energy storage system according to one embodiment of the present disclosure.

FIG. 8 is a perspective view illustrating an example of a branch pipe and an extinguishing nozzle according to one embodiment of the present disclosure.

FIG. 9 is a perspective view illustrating an example of a heat-sensitive member included in an extinguishing nozzle according to one embodiment of the present disclosure.

FIG. 10 is a cross-sectional view illustrating an example in which an extinguishing agent is supplied to a battery cell through an extinguishing valve according to one embodiment of the present disclosure.

### DETAILED DESCRIPTION

Hereinafter, embodiments of the present disclosure will be described, in detail, with reference to the accompanying drawings. The terms or words used in this specification and claims should not be construed as being limited to the usual or dictionary meaning and should be interpreted as meaning and concept consistent with the technical idea of the present disclosure based on the principle that the inventor can be his/her own lexicographer to appropriately define the concept of the term to explain his/her invention in the best way.

The embodiments described in this specification and the configurations shown in the drawings are only some of the embodiments of the present disclosure and do not represent all of the technical ideas, aspects, and features of the present disclosure. Accordingly, it should be understood that there may be various equivalents and modifications that can replace or modify the embodiments described herein at the time of filing this application.

It will be understood that when an element or layer is referred to as being “on,” “connected to,” or “coupled to” another element or layer, it may be directly on, connected, or coupled to the other element or layer or one or more intervening elements or layers may also be present. When an element or layer is referred to as being “directly on,” “directly connected to,” or “directly coupled to” another element or layer, there are no intervening elements or layers present. For example, when a first element is described as being “coupled” or “connected” to a second element, the first element may be directly coupled or connected to the second element or the first element may be indirectly coupled or connected to the second element via one or more intervening elements.

In the figures, dimensions of the various elements, layers, etc. may be exaggerated for clarity of illustration. The same reference numerals designate the same elements. As used herein, the term “and/or” includes any and all combinations of one or more of the associated listed items. Further, the use of “may” when describing embodiments of the present disclosure relates to “one or more embodiments of the present disclosure.” Expressions, such as “at least one of” and “any one of,” when preceding a list of elements, modify the entire list of elements and do not modify the individual elements of the list. When phrases such as “at least one of A, B and C, “at least one of A, B or C,” “at least one selected from a group of A, B and C,” or “at least one selected from among A, B and C” are used to designate a list of elements A, B and C, the phrase may refer to any and all suitable combinations or a subset of A, B and C, such as A, B, C, A and B, A and C, B and C, or A and B and C. As used herein, the terms “use,” “using,” and “used” may be considered synonymous with the terms “utilize,” “utilizing,” and “utilized,” respectively. As used herein, the terms “substantially,” “about,” and similar terms are used as terms of approximation and not as terms of degree, and are intended to account for the inherent variations in measured or calculated values that would be recognized by those of ordinary skill in the art.

It will be understood that, although the terms first, second, third, etc. may be used herein to describe various elements, components, regions, layers, and/or sections, these elements, components, regions, layers, and/or sections should not be limited by these terms. These terms are used to distinguish one element, component, region, layer, or section from another element, component, region, layer, or section. Thus, a first element, component, region, layer, or section discussed below could be termed a second element, component, region, layer, or section without departing from the teachings of example embodiments.

Spatially relative terms, such as “beneath,” “below,” “lower,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” or “over” the other elements or features. Thus, the term “below” may encompass both an orientation of above and below. The device may be otherwise oriented (rotated 90 degrees or at other orientations), and the spatially relative descriptors used herein should be interpreted accordingly.

The terminology used herein is for the purpose of describing embodiments of the present disclosure and is not intended to be limiting of the present disclosure. As used herein, the singular forms “a” and “an” are intended to include the plural forms as well, unless the context clearly indicates otherwise. It will be further understood that the terms “includes,” “including,” “comprises,” and/or “comprising,” when used in this specification, specify the presence of stated features, integers, steps, operations, elements, and/or components but do not preclude the presence or addition of one or more other features, integers, steps, operations, elements, components, and/or groups thereof.

Also, any numerical range disclosed and/or recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein, and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein. All such ranges are intended to be inherently described in this specification such that amending to expressly recite any such subranges would comply with the requirements of 35 U.S.C. § 112(a) and 35 U.S.C. § 132(a).

References to two compared elements, features, etc. as being “the same” may mean that they are “substantially the same”. Thus, the phrase “substantially the same” may include a case having a deviation that is considered low in the art, for example, a deviation of 5% or less. In addition, when a certain parameter is referred to as being uniform in a given region, it may mean that it is uniform in terms of an average.

Throughout the specification, unless otherwise stated, each element may be singular or plural.

Arranging an arbitrary element “above (or below)” or “on (under)” another element may mean that the arbitrary element may be disposed in contact with the upper (or lower) surface of the element, and another element may also be interposed between the element and the arbitrary element disposed on (or under) the element.

In addition, it will be understood that when a component is referred to as being “linked,” “coupled,” or “connected” to another component, the elements may be directly “coupled,” “linked” or “connected” to each other, or another component may be “interposed” between the components”.

A battery pack according to one or more embodiments includes at least one battery module and a pack housing having an accommodation space in which the at least one battery module is accommodated.

The battery module may include a plurality of battery cells and a module housing. The battery cells may be accommodated inside the module housing in a stacked form (or stacked arrangement or configuration). Each battery cell may have a positive electrode terminal and a negative electrode terminal and may be a circular type, a prismatic type, or a pouch type according to the shape of battery. In the present specification, a battery cell may also be referred to as a secondary battery, a battery, or a cell.

In the battery pack, one cell stack may constitute one module stacked in place of the battery module. The cell stack may be accommodated in an accommodation space of the pack housing or may be accommodated in an accommodation space partitioned by a frame, a partition wall, etc.

During the discharging and/or charging of rechargeable batteries, heat generation may occur. If the heating persists, thermal runaway of the rechargeable batteries may occur, potentially leading to fire in devices or systems containing the rechargeable batteries. Additionally, the generated heat may be accumulated in the battery cell, thereby accelerating the deterioration of the battery cell. Accordingly, the battery pack may further include a cooling member to remove the generated heat and thereby suppress deterioration of the battery cell. The cooling member may be provided at the bottom of the accommodation space at where the battery cell is provided but is not limited thereto and may be provided at the top or side depending on the battery pack.

The battery cell may be configured such that exhaust gas generated inside the battery cell under abnormal operating conditions, also known as thermal runaway or thermal events, is discharged to the outside of the battery cell. The battery pack or the battery module may include an exhaust port for discharging the exhaust gas to prevent or reduce damage to the battery pack or module by the exhaust gas.

The battery pack may include a battery and a battery management system (BMS) for managing the battery. The battery management system may include a detection device, a balancing device, and a control device. The battery module may include a plurality of cells connected to each other in series and/or parallel. The battery modules may be connected to each other in series and/or in parallel.

The detection device may detect a state of a battery (e.g., voltage, current, temperature, etc.) to output state information indicating the state of the battery. The detection device may detect the voltage of each cell constituting the battery or of each battery module. The detection device may detect current flowing through each battery module constituting the battery module or the battery pack. The detection device may also detect the temperature of a cell and/or module on at least one point of the battery and/or an ambient temperature.

The balancing device may perform a balancing operation of a battery module and/or cells constituting the battery module. The control device may receive state information (e.g., voltage, current, temperature, etc.) of the battery module from the detection device. The control device may monitor and calculate the state of the battery module (e.g., voltage, current, temperature, state of charge (SOC), life span (state of health (SOH)), etc.) on the basis of the state information received from the detection device. In addition, on the basis of the monitored state information, the control device may perform a control function (e.g., temperature control, balancing control, charge/discharge control, etc.) and a protection function (e.g., over-discharge, over-charge, over-current protection, short circuit, fire extinguishing function, etc.). In addition, the control device may perform a wired or wireless communication function with an external device of the battery pack (e.g., a higher level controller or vehicle, charger, power conversion system, etc.).

The control device may control charging/discharging operation and protection operation of the battery. To this end, the control device may include a charge/discharge control unit, a balancing control unit, and/or a protection unit.

The battery management system is a system that monitors the battery state and performs diagnosis and control, communication, and protection functions, and may calculate the charge/discharge state, calculate battery life or state of health (SOH), cut off, as necessary, battery power (e.g., relay control), control thermal management (e.g., cooling, heating, etc.), perform a high-voltage interlock function, and/or may detect and/or calculate insulation and short circuit conditions.

A relay may be a mechanical contactor that is turned on and off by the magnetic force of a coil or a semiconductor switch, such as a metal oxide semiconductor field effect transistor (MOSFET).

The relay control has a function of cutting off the power supply from the battery if (or when) a problem occurs in the vehicle and the battery system and may include one or more relays and pre-charge relays at the positive terminal and the negative terminal, respectively.

In the pre-charge control, there is a risk of inrush current occurring in the high-voltage capacitor on the input side of the inverter when the battery load is connected. Thus, to prevent inrush current when starting a vehicle, the pre-charge relay may be operated before connecting the main relay and the pre-charge resistor may be connected.

The high-voltage interlock is a circuit that uses a small signal to detect whether or not all high-voltage parts of the entire vehicle system are connected and may have a function of forcibly opening a relay if (or when) an opening occurs at even one location on the entire loop.

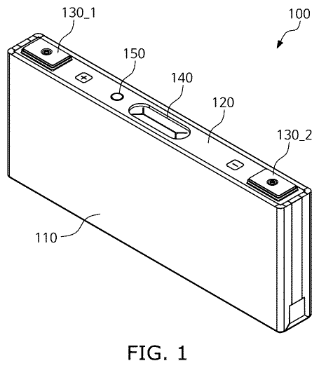

FIG. 1 illustrates a perspective view showing an example of a battery cell 100 according to embodiments of the present disclosure. Referring to FIG. 1, the battery cell 100 may include: one or more electrode assemblies wound or stacked, with a separator, i.e., an insulator, being provided between a positive electrode and a negative electrode; a casing 110 including the electrode assemblies disposed therein; and a cap plate 120 coupled to one open end of the casing 110. The battery cell 100 shown in FIG. 1 may be a secondary battery.

The positive electrode and the negative electrode may include a current collector made of a thin metal foil having a coated portion on which an active material is coated and an uncoated portion on which an active material is not coated.

The positive electrode and the negative electrode may be wound after interposing a separator, which is an insulator, therebetween. However, the present disclosure is not limited thereto, and the electrode assembly described above may have a structure in which a positive electrode and a negative electrode, each made of a plurality of sheets, are alternately stacked with a separator interposed therebetween.

The casing 110 may form the overall appearance of the battery cell 100 and may be formed of a conductive metal such as aluminum, an aluminum alloy, or nickel-plated steel. The casing 110 may also provide a space in which the electrode assembly is accommodated.

In FIG. 1, the casing 110 is shown as being a prismatic casing and the battery cell 100 is shown as being an angled battery cell, but the scope of the present disclosure is not limited thereto. The battery cell 100 may be any shape battery cell, such as a prismatic battery cell, a cylindrical battery cell, or a pouch-shaped battery cell.

The cap plate 120 may be coupled to the open end of the casing 110 to seal the casing 110. The casing 110 and the cap plate 120 may include a conductive material. In some embodiments, the upper end of the casing 110 may be open, and the cap plate 120 may seal the open upper end of the casing 110. A positive electrode terminal 130_1 electrically connected to the positive electrode and a negative electrode terminal 130_2 electrically connected to the negative electrode may be coupled to the cap plate 120. For example, the positive and negative electrode terminals 130_1 and 1302 may be disposed to protrude outward through the cap plate 120.

In some embodiments, a vent 140 may be provided in at least one surface of the battery cell 100 (e.g., the upper surface of the battery cell 100, i.e., the cap plate 120, in the shown example). The vent 140 may be configured to be opened in a case where an internal pressure equal to or higher than a predetermined threshold pressure is detected in the battery cell 100. The vent 140 may serve as a passage for venting vent gases generated inside the battery cell 100.

In some embodiments, the cap plate 120 may include an electrolyte inlet 150. For example, the electrolyte inlet 150 may be a through-hole provided in the cap plate 120. The electrolyte inlet 150 may be formed to allow electrolyte to be injected into the casing 110 therethrough after the cap plate 120 is coupled to the opening of the casing 110 to seal the casing 110. The electrolyte inlet 150 may be sealed with a sealing member after the injection of the electrolyte. The battery cell 100 may be a lithium (Li) battery cell, a sodium (Na) battery cell, or the like. However, the scope of the present disclosure is not limited thereto, and the battery cell 100 includes any battery able to repeatedly provide electricity due to charge and discharge.

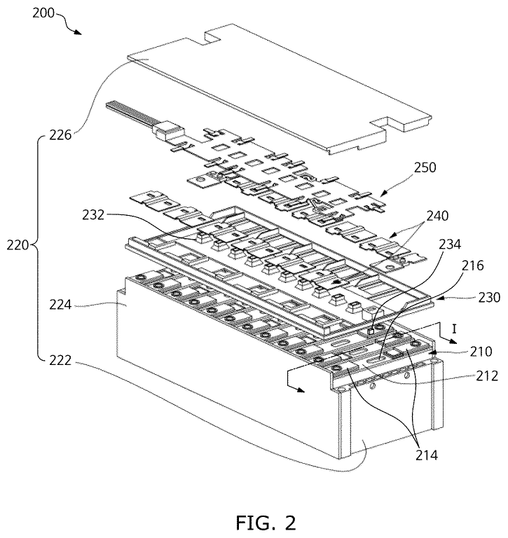

FIG. 2 is an exploded perspective view showing an example in which a part of a battery module 200 is disassembled according to one embodiment of the present disclosure. Referring to FIG. 2, the battery module 200 according to one embodiment of the present disclosure may include a plurality of battery cells 210, a frame 220 accommodating the plurality of battery cells 210, a plurality of busbars 240 electrically connected to the plurality of battery cells 210, a busbar holder 230 supporting the plurality of busbars 240, and circuitry 250 electrically connected to the plurality of busbars 240 and including various circuits and components. In one embodiment, the battery module 200 may also be referred to as a battery pack. Further, in one embodiment, the battery module 200 may be included in a vehicle, an energy storage system (ESS), or the like.

The battery module 200 may include a plurality of battery cells 210. In one embodiment, in the battery module 200, the plurality of battery cells 210 may be arranged in one direction such that their wide surfaces face each other. FIG. 2 shows that the plurality of battery cells are arranged in a row within the battery module 200. However, the scope of the present disclosure is not limited thereto, and the plurality of battery cells may be arranged in multiple columns.

In one embodiment, each of the battery cells 210 may include a casing, an electrode assembly accommodated together with an electrolyte in the casing, and a cap plate 212 for sealing the casing. The electrode assembly may be formed by sequentially winding or stacking a negative electrode plate, a separator, and a positive electrode plate. A negative electrode active material such as graphite or carbon may be applied or coated on the negative electrode plate formed of a metal foil such as copper, copper alloy, nickel, or nickel alloy. An active material such as a transition metal oxide may be applied or coated on the positive electrode plate formed of a metal foil such as aluminum or aluminum alloy. Each of the negative electrode plate and the positive electrode plate may include an uncoated portion that is a region where no active material is applied. A negative electrode tab may be connected to a negative electrode uncoated portion, and a positive electrode tab may be connected to a positive electrode uncoated portion. Further, the negative and positive electrode tabs may be electrically connected to negative and positive electrode terminals 214 formed on the cap plate 212, respectively. The negative and positive electrode terminals 214 formed on the cap plate 212 may be electrically connected to the busbar 240. However, the structure of the battery cell 210 is not limited thereto, and may be appropriately modified if necessary. Furthermore, the number and arrangement of the battery cells 210 are not limited to the structure shown in FIG. 2 and may be appropriately modified if necessary.

The plurality of battery cells 210 may be accommodated in the frame 220. In one embodiment, the frame 220 may include a pair of end plates 222 that contact the outermost battery cells 210 in a direction of the arrangement of the battery cells 210, a pair of side plates 224 that are orthogonally coupled to the end plates 222, and a top plate 226 that is disposed above the circuitry 250. Although not shown, a bottom plate may be disposed below the battery cells 210 to support the battery cells 210 from the bottom. The frame 220 may accommodate the battery cells 210, the busbar holder 230, the busbars 240, and the circuitry 250.

In FIG. 2, the busbar holder 230 may be disposed above the cap plate 212 and may support the busbars 240. For example, the busbar holder 230 may be a substantially rectangular panel made of an insulating material.

A plurality of through-holes through which the positive and negative electrode terminals 214 of the cap plate 212 are exposed may be formed in the busbar holder 230. The positive and negative electrode terminals 214 may be exposed through the through-holes formed in the busbar holder 230, and the busbars 240 may be electrically connected to the exposed positive and negative electrode terminals 214.

A plurality of passageways 232 may be formed through the busbar holder 230. The passageways 232 may be formed to penetrate the busbar holder 230. In this case, the passageways 232 may be formed at positions corresponding to vents 216 of the battery cells 210, respectively. Accordingly, the passageways 232 may serve as passages for vent gas discharged from the vents 216 to flow therethrough. Further, the passageways 232 may also serve as passages for the movement of extinguishing agents during the operation of a fire extinguishing system. In FIG. 2, the shape of each passageway 232 is shown as a roughly square column. However, the scope of the present disclosure is not limited thereto, and each passageway may be formed in various shapes, such as a cylindrical shape, a polygonal column shape, a narrow and elongated slit shape, or the like. For example, the shape of each passageway 232 may be determined based on the shape of each vent 216.

A plurality of gas flow detection sensors 234 may be coupled to the busbar holder 230. The gas flow detection sensors 234 may be fixedly coupled to a bottom surface of the busbar holder 230 that faces the battery cells 210 so as to respectively correspond to the battery cells 210. The gas flow detection sensors 234 may be disposed at positions corresponding to the vents 216 of the battery cells 210. For example, the gas flow detection sensors 234 may be disposed near or within the passageways 232, respectively.

For each battery cell, the gas flow detection sensor 234 may be positioned corresponding to the vent 216 of the battery cell 210 to detect a flow of gas discharged from the vent 216. For example, the gas flow detection sensor 234 may detect at least one of a wind pressure, a wind speed, or an air volume of the vent gas discharged from the vent 216. In response to a case where at least one of the detected wind pressure, wind speed, or air volume is greater than or equal to a predetermined threshold, the gas flow detection sensor 234 may determine that the flow of gas has been detected. The busbar holder 230 may have a plurality of engagement portions formed thereon for engaging and securing the plurality of gas flow detection sensors 234. In another example, the plurality of gas flow detection sensors 234 may be fixedly coupled to a bottom surface of the top plate 226 to respectively correspond to the plurality of battery cells 210.

The busbars 240 may electrically connect the positive and negative electrode terminals 214. The plurality of busbars 240 are provided in order to connect the plurality of battery cells 210 in series and/or parallel. In one embodiment, each busbar 240 may electrically connect the positive electrode terminal of one of the battery cells 210 with the positive electrode terminal 214 or the negative electrode terminal 214 of another one of the battery cells 210. Additionally or alternatively, each busbar 240 may electrically connect the negative electrode terminal of one of the battery cells 210 with the positive electrode terminal 214 or the negative electrode terminal 214 of another one of the battery cells 210. The busbars 240 may be connected to the positive electrode terminals 214 and/or the negative electrode terminals 214 by welding, for example. Regions of each battery cell 210 other than the positive and negative electrode terminals 214 of the battery cell 210 may be insulated from the busbar 240 by the busbar holder 230.

The circuitry 250 may be disposed between the busbars 240 and the top plate 226, and the busbars 240 and the gas flow detection sensors 234 may be electrically connected with the circuitry 250. In one embodiment, the circuitry 250 may have a substantially rectangular shape and may be disposed above the busbar holder 230. The circuitry 250 may be arranged to be adjacent to at least the installation area of the busbars 240 to facilitate a smooth connection with the busbars 240.

The circuitry 250 may include various components for measuring status information of the battery cells 210, such as voltages and/or temperatures of the battery cells 210, and various components or circuits for controlling and/or managing the battery cells 210. The circuitry 250 may be electrically connected to the outside of the battery module 200 via separate connectors.

In one embodiment, the circuitry 250 may include a battery management module (BMM). The battery management module may be configured to monitor the status of the plurality of battery cells 210 included in the battery module 200 by monitoring, for example, voltages, currents, temperatures of the battery cells 210 and managing battery charging and discharging. In another example, the battery management module (BMM) may not be included in the circuitry 250, but may be separately disposed within the battery module 200. For example, the battery management module (BMM) may be attached to the end plate 222.

The battery management module (BMM) that monitors the battery module 200 may be connected to a battery management system (BMS). In one embodiment, the battery management system (BMS) may be connected to a plurality of battery management modules (BMMs). For example, the battery management system and the plurality of battery management modules (BMMs) may be interconnected in a daisy-chain connection. That is, the plurality of battery management modules (BMMs) that respectively monitoring the plurality of battery modules 200 may be managed collectively by the battery management system (BMS). Accordingly, the battery management system (BMS) may monitor an entire energy storage system (ESS) (not shown) including the plurality of battery modules 200.

In one embodiment, the gas flow detection sensors 234 may be electrically connected to the battery management module (BMM) included in the circuitry 250. In one embodiment, the gas flow detection sensor 234 may transmit a gas flow detection signal to the battery management module (BMM) in response to the detection of gas flow. In this case, the battery management module (BMM) may receive the gas flow detection signal and transmit an extinguishing preparation signal to the battery management system (BMS). The operation of the gas flow detection sensor 234 and the first extinguishing system utilizing the same will be described in more detail in subsequent diagrams.

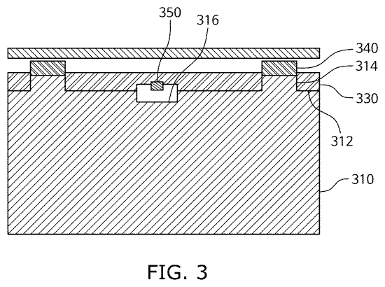

FIG. 3 is a cross-sectional view illustrating a gas flow detection sensor 350 coupled to a busbar holder 330 according to one embodiment of the present disclosure. FIG. 3 is a partial cross-sectional view taken along a line I of FIG. 2. According to the example shown in FIG. 3, electrode terminals 314 and a vent 316 may be formed at a top surface of a battery cell 310, that is, a cap plate 312. A busbar holder 330 may be disposed above the battery cell 310. The electrode terminals 314 may be exposed through through-holes formed in the busbar holder 330, and busbars 340 may be electrically connected to the exposed electrode terminals 314. Although not shown, circuitry (e.g., the circuitry 250 shown in FIG. 2) may be disposed above the busbar holder 330.

A gas flow detection sensor 350 may be coupled to a bottom surface of the busbar holder 330. The gas flow detection sensor 350 may be disposed at a position corresponding to the vent 316 of the battery cell 310. In addition, the busbar holder 330 may have a passageway (e.g., the passageway 232 shown in FIG. 2) for venting vent gas discharged from the vent 316. In this case, the gas flow detection sensor 350 may detect the vent gas flowing through the passageway. The passageway may also serve as a passage for the movement of extinguishing agents during the operation of a fire extinguishing system. The gas flow detection sensor 350 may be disposed near or within the passageway.

The gas flow detection sensor 350 may be disposed at every predetermined number of battery cells 310 to monitor each of the predetermined number of battery cells 310. For example, one gas flow detection sensor 350 may be disposed for every battery cell 310 (one-to-one relationship), or one gas flow detection sensor 350 may be disposed for every two neighboring battery cells 310 (one-to-two relationship). The gas flow detection sensor 350 may detect the flow of gas associated with the one or more battery cells 310 monitored by the gas flow detection sensor 350.

The gas flow detection sensor 350 may detect a flow of vent gas that is generated inside the battery cell 310 and discharged through the vent 316. The gas flow detection sensor 350 may detect at least one of a wind speed, a wind pressure, or an air volume of the vent gas. In response to a case where at least one of the wind speed, the wind pressure, or the air volume detected by the gas flow detection sensor 350 is greater than or equal to a predetermined threshold, the gas flow detection sensor 350 may transmit a gas flow detection signal to the battery management module (BMM). Thereafter, the battery management module (BMM) may transmit the gas flow detection signal to the battery management system (BMS).

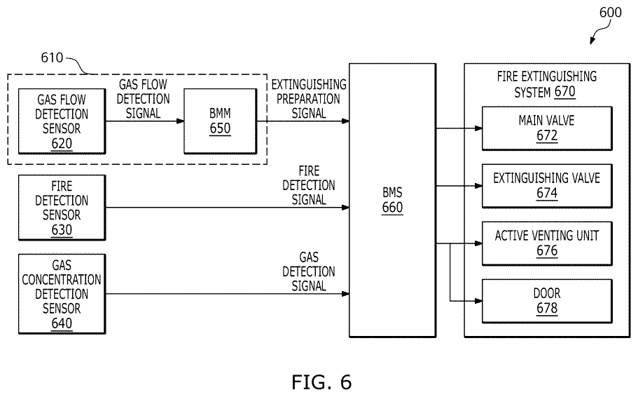

In another example, the gas flow detection sensor 350 may detect at least one of the wind speed, the wind pressure, or the air volume in real time and continuously transmit the detection data to the battery management module (BMM). In this case, the battery management module (BMM) may determine the occurrence of gas flow based on the detection data and transmit a gas flow detection signal to the battery management system (BMS). An example of a method of transmitting the gas flow detection signal will be described in more detail in FIG. 6.

With this configuration, the plurality of gas flow detection sensors 350 may allow individual monitoring of each of the battery cells 310. Consequently, by detecting, among the battery cells 310, specific battery cells 310 exhibiting abnormal behaviors such as vent gas discharge at an early stage, it allows for the accurate and rapid response to the abnormal behaviors in the specific battery cells 310. Further, by detecting the flow of vent gas before events such as fire occurring in a particular battery cell 310 spreads throughout the entire battery module, it allows for rapid fire response.

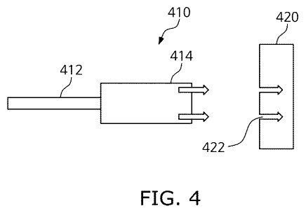

FIG. 4 is a diagram illustrating an example in which a gas flow detection sensor 410 is fastened to a busbar holder 420 according to one embodiment of the present disclosure. The gas flow detection sensor 410 may include a sensor body 412 and a first engagement portion 414. In one embodiment, the first engagement portion 414 may be formed to protrude from the sensor body 412.

In one embodiment, the first engagement portion 414 of the gas flow detection sensor 410 may be in the form of at least one hook protruding from one surface of the sensor body 412. For example, as shown in FIG. 4, the first engagement portion 414 may be formed to protrude from one side of the sensor body 412. Alternatively or additionally, the first engagement portion 414 may be formed to project from another side, for example, at least one of a top surface or a bottom surface of the sensor body 412.

In the example shown in FIG. 4, the first engagement portion 414 has two hooks that are formed in parallel, but the number and arrangement of the hooks are not limited to the structure shown in FIG. 4 and may be appropriately changed, if necessary.

The busbar holder 420 may include a second engagement portion 422 to which the gas flow detection sensor 410 is fixedly coupled. The second engagement portion 422 may be formed at a bottom surface of the busbar holder 420 facing the battery cell. Specifically, the second engagement portion 422 may be formed near a position corresponding to a vent of the battery cell.

The second engagement portion 422 of the busbar holder 420 may be formed in a groove shape to receive the first engagement portion 414 of the gas flow detection sensor 410. In this case, the first engagement portion 414 of the gas flow detection sensor 410 may be inserted into and engaged with the second engagement portion 422 of the busbar holder 420. Accordingly, the gas flow detection sensor 410 may be fixedly coupled at the bottom surface of the busbar holder 420.

As described above, the position of the second engagement portion 422 and the number and arrangement of the grooves of the second engagement portion 422 are not limited to the structure shown in FIG. 4, and may be appropriately changed if necessary. That is, the second engagement portion 422 may be positioned to correspond to the position of the first engagement portion, and the number and arrangement of the grooves formed at the second engagement portion 422 may be determined to correspond to the number and arrangement of the hooks formed at the first engagement portion 414. Furthermore, the engagement structure between the gas flow detection sensor 410 and the busbar holder 420 is not limited to the structure shown in FIG. 4, and any structure that allows the gas flow detection sensor 410 to be fixedly coupled to the busbar holder 420 can be employed. For example, the first engagement portion 414 of the gas flow detection sensor 410 may be in the form of a groove, and the second engagement portion 422 of the busbar holder 420 may be in the form of a hook.

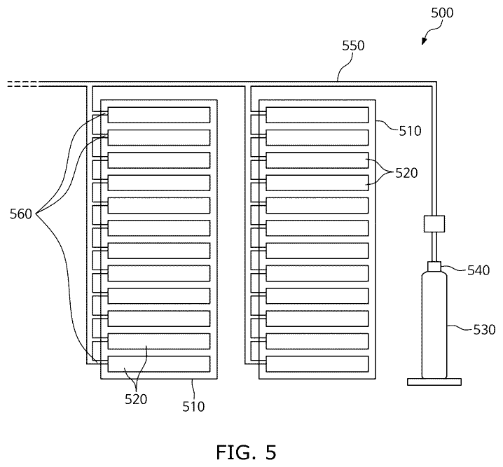

FIG. 5 is a diagram illustrating an example of a fire extinguishing system included in an energy storage system 500 according to one embodiment of the present disclosure. In one embodiment, the energy storage system 500 may include a fire extinguishing system to respond to an event (e.g., a fire) occurring in a battery cell. Before describing the fire extinguishing system, the energy storage system 500 will be briefly described.

In one embodiment, the energy storage system 500 may include a plurality of battery modules 520, and at least one battery rack 510 in which the plurality of battery modules 520 are accommodated. A plurality of battery cells may be accommodated within a casing of each of the battery modules 520.

The energy storage system 500 may include the battery management system (BMS). The battery management system (BMS) may be connected to a plurality of battery management modules (BMMs) included in each of the plurality of battery modules 520. For example, the battery management system and the plurality of battery management modules may be interconnected in a daisy-chain connection. That is, the battery management system may collectively monitor and manage all of the plurality of battery modules 520 included in the energy storage system 500.

The energy storage system 500 may include a fire extinguishing system. In order to respond to events such as vent gas discharge, fire outbreak and the like occurring in a plurality of battery cells included in the energy storage system 500, the battery management system (BMS) may activate the fire extinguishing system in response to detection signals and/or detection data for each of the battery cells.

The fire extinguishing system may include an agent container 530, a main valve 540, a main pipe 550, and a branch pipe 560. The agent container 530 may be a type of storage vessel that stores an extinguishing agent. The agent container 530 may be a pressure vessel that stores a high-pressure extinguishing agent. The extinguishing agent may be stored in the agent container 530 using either a stored-pressure method or a cartridge-operated method. Accordingly, upon activation for the discharge of the extinguishing agent from the high-pressure agent container 530, the main valve 540 may be opened to discharge the extinguishing agent.

The main valve 540 may function to open and close the discharge portion of the agent container 530. The main valve 540 may control the supply or the interruption of supply of the extinguishing agent in response to instructions from the battery management system (BMS). In response to the main valve 540 being opened, the extinguishing agent may be discharged from the agent container 530 and transferred through the main pipe 550.

The main pipe 550 may extend from the agent container 530 to transfer the extinguishing agent toward each of the battery racks 510. The main pipe 550 may be disposed and installed adjacent to the exterior of each of the battery racks 510. As shown in FIG. 5, the main pipe 550 may be installed adjacent to the battery racks 510 while extending in a direction parallel to a direction in which the plurality of battery modules 520 are aligned inside each of the battery racks 510. In another example, the main pipe 550 may be branched and installed to be inserted into the interior of each of the plurality of battery racks 510. In this case, the branched main pipe 550 may be installed in an upper region within each of the battery racks 510 while extending in the direction parallel to the alignment direction of the plurality of battery modules 520 inside each of the battery racks 510.

The main pipe 550 may be branched into branch pipes 560. The branch pipes 560 may be respectively connected to the plurality of battery modules 520 included in each of the battery racks 510. The branch pipe 560 may be installed in an upper region within each of the battery modules 520 while extending in a direction parallel to a direction in which the battery cells are aligned inside each of the battery modules 520.

The number of branch pipes 560 connected to each battery module 520 may correspond to the number of columns of battery cells stored in the battery module 520. For example, in a case where battery cells are arranged in two columns within one battery module 520, two branch pipes 560 may be connected to the battery module 520. That is, at least one branch pipe 560 may be installed in each column where battery cells are arranged.

The branch pipe 560 connected to the battery module 520 may be disposed above the individual battery cells. The extinguishing agent transferred through the branch pipe 560 may be sprayed directly onto the top of the battery cell (e.g., the vent of the battery cell) through an extinguishing nozzle (not shown) formed in the branch pipe 560.

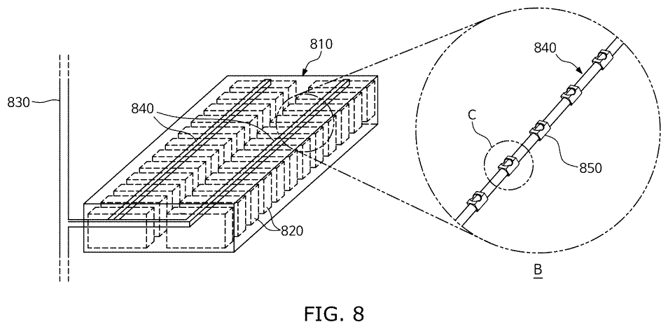

In one embodiment, the extinguishing nozzle may include a heat-sensitive member. In this case, the extinguishing nozzle may melt at a predetermined threshold temperature, allowing the extinguishing agent transferred through the branch pipe 560 to be sprayed onto the top of the battery cells. In another embodiment, the extinguishing nozzle may include an extinguishing valve. In this case, the extinguishing valve may be controlled to open and close by the battery management system (BMS). Thus, the extinguishing agent transferred through the branch pipe 560 may be sprayed onto the top of the battery cells in response to instructions from the battery management system (BMS). Examples in which the extinguishing agent is sprayed through the extinguishing nozzle will be described in more detail in FIGS. 8 to 10.

With this configuration, the extinguishing agent discharged from the agent container 530 can be transferred through the main pipe 550 and the branch pipes 560 to reach all of the battery cells included in the energy storage system 500. Accordingly, the extinguishing agent can be sprayed intensively onto battery cells exhibiting abnormal behavior (for example, exhibiting vent gas discharge, fire outbreak, and the like).

FIG. 6 is a block diagram illustrating an operational flow of a fire extinguishing system 670 according to one embodiment of the present disclosure. The energy storage system 600 may detect abnormal behavior in the battery cells using a plurality of detection sensors 620 to 640. The plurality of detection sensors 620 to 640 may transmit abnormality detection signals and/or abnormality detection data for the battery cells to a battery management system (BMS) 660. In this case, the battery management system (BMS) 660 may activate the fire extinguishing system 670 to respond to the cause of the abnormality detection signals and/or the abnormality detection data.

The energy storage system 600 may include gas flow detection sensors 620. The gas flow detection sensors 620 may be disposed to correspond with individual battery cells included in a battery module 610. Each of the gas flow detection sensors 620 may be disposed to detect gas flow from one or more battery cells.

Each gas flow detection sensor 620 may detect a flow of vent gas that is generated inside the battery cell and expelled through the vent. The gas flow detection sensor 620 may detect at least one of a wind speed, a wind pressure, or an air volume of the vent gas. In response to a case where at least one of the wind speed, the wind pressure, or the air volume detected by the gas flow detection sensor 620, the gas flow detection sensor 620 may transmit a gas flow detection signal to a battery management module (BMM) 650.

The energy storage system 600 may include a fire detection sensor 630. In one embodiment, the fire detection sensor 630 may function as a smoke detection sensor. Considering the upward movement of smoke, the fire detection sensor 630 may be installed in an upper area of each of the battery racks. However, the installation location and the number of the fire detection sensors 630 are not limited thereto, and the installation location and the number of the fire detection sensors 630 may be adjusted as necessary. For example, additional fire detection sensors 630 may be installed in a lower area of the battery rack or in a space between the battery racks. Upon detecting a fire (e.g., smoke), the fire detection sensor(s) 630 transmits a fire detection signal to the battery management system (BMS) 660.

The energy storage system 600 may include a gas concentration detection sensor 640. In one embodiment, the gas concentration detection sensor 640 may detect gases (e.g., CO, CO2, HCN, HCl, and the like) generated during a fire. The gas concentration detection sensor 640 may be installed inside the energy storage system 600, inside the battery rack, and/or inside the battery module. The gas concentration detection sensor 640 may transmit a gas detection signal to the battery management system (BMS) 660 in response to detecting a gas concentration equal to or greater than a predetermined threshold.

Additionally, the energy storage system 600 may further include a temperature sensor or the like.

The energy storage system 600 may include a fire extinguishing system 670. As described above in FIG. 5, the fire extinguishing system 670 may include a main valve 672 for supplying or stopping the supply of extinguishing agent, a main pipe (e.g., the main pipe 550 shown in FIG. 5) and branch pipes (e.g., the branch pipes 560 shown in FIG. 5) for transferring the extinguishing agent from an agent container to a battery cell, and an extinguishing nozzle that is a passage through which the extinguishing agent is sprayed onto the battery cell. The extinguishing nozzle may include a heat-sensitive member or an extinguishing valve 674. In FIG. 6, the operational flow of the fire extinguishing system 670 is described based on an example in which the extinguishing nozzle includes the extinguishing valve 674.

Additionally, the fire extinguishing system 670 may include an active venting unit 676 for venting vent gases diffused within the energy storage system 600 to the outside. The active venting unit 676 may be formed through the housing of the energy storage system 600 to communicate between the interior and exterior of the energy storage system 600. The active venting unit 676 may be formed at an upper portion of the energy storage system 600, depending on the nature of the gas being vented. The battery management system (BMS) 660 may control the active venting unit 676 to be activated in response to detecting a gas concentration equal to or greater than a predetermined threshold.

Additionally, the fire extinguishing system 670 may include a door 678 of the energy storage system 600 that is controlled to open in the event of a fire. The door 678 may be configured to open and close one side of the energy storage system 600. The battery management system (BMS) 660 may control the door 678 to be opened in response to detecting a gas concentration equal to or greater than a predetermined threshold.

In one embodiment, if a particular battery cell within the energy storage system 600 exhibits abnormal behavior (e.g., vent gas discharge), the gas flow detection sensor 620 may be the first to detect the abnormality. The gas flow detection sensor 620 may detect a flow of vent gas discharged from a particular battery cell. In response to a case where at least one of the wind speed, the wind pressure, or the air volume detected by the gas flow detection sensor 620 is equal to or greater than a predetermined threshold, the gas flow detection sensor 620 may transmit a gas flow detection signal to the battery management module (BMM) 650.

Thereafter, the battery management module (BMM) 650 may, in response to receiving the gas flow detection signal, transmit an extinguishing preparation signal to the battery management system (BMS) 660. The extinguishing preparation signal may include information associated with the battery cell in which the flow of gas is detected (e.g., identifier information of the battery cell, location information of the battery cell, and the like). In response to receiving the extinguishing preparation signal, the battery management system (BMS) 660 may control the main valve 672 included in the fire extinguishing system 670 to be opened. In response to the main valve 672 being opened, the extinguishing agent may be discharged from the agent container and transferred into the battery module 610 through the main pipe and the branch pipes. Accordingly, the extinguishing agent may be in a state where it is ready to be sprayed into the battery cells immediately.

After the gas flow detection sensor 620 detects the flow of gas, smoke may be detected by the fire detection sensor 630 if a fire occurs in the battery cell. In a case where the fire detection sensor 630 detects the smoke, the fire detection sensor 630 may transmit a fire detection signal to the battery management system (BMS) 660. In response to receiving the fire detection signal, the battery management system (BMS) 660 may control a particular extinguishing valve 674 included in the fire extinguishing system 670 to be opened. Here, the particular extinguishing valve 674 may be associated with a particular battery cell in which the flow of gas was detected. Consequently, upon opening of the particular extinguishing valve 674, the extinguishing agent may be immediately sprayed onto and injected into the particular battery cell.

The opening of the extinguishing valve 674 may occur after the main valve 672 is opened by the battery management system 660 in response to the extinguishing preparation signal. That is, the battery management system 660 may initiate the transfer of the extinguishing agent from the agent container in response to the extinguishing preparation signal before the extinguishing valve 674 is opened. With this configuration, the transfer of the extinguishing agent may be initiated before smoke is detected by the fire detection sensor 630, thereby reducing the fire response time by the amount of time difference between the gas flow detection time via the gas flow detection sensor 620 and the smoke detection time via the fire detection sensor 630.

Subsequent to the gas flow detection by the gas flow detection sensor 620, either simultaneously or after a certain period of time, gas may be detected by the gas concentration detection sensor 640. In a case where the gas concentration detection sensor 640 detects a gas concentration equal to or greater than a threshold value, a gas detection signal may be transmitted to the battery management system (BMS) 660. In response to receiving the gas detection signal, the battery management system (BMS) 660 may control the active venting unit 676 to be activated. Accordingly, vent gas that has diffused inside the energy storage system 600 may be vented to the outside.

Additionally, in response to receiving the gas detection signal, the battery management system (BMS) 660 may control a door that opens or closes one side of the energy storage system 600 to be opened. In response to the door being opened, the battery management system (BMS) 660 may transmit a door opening notification to an external device. As the door is opened, the air inside the energy storage system 600 can be vented to the outside more quickly, and any necessary follow-up actions, such as fire suppression, can be carried out more quickly.

In one embodiment, the battery management system (BMS) 660 may transmit an alert signal to an external device in response to receiving at least one of an extinguishing preparation signal, a fire detection signal, or a gas detection signal. For example, the battery management system (BMS) may transmit an evacuation signal to an external device. Here, the external device may include speakers, sirens, or other devices installed inside and/or outside the energy storage system 600. Further, the external device may include a control system, an administrator terminal, and similar devices that control the energy storage system 600.

In one embodiment, the gas flow detection sensor 620, the fire detection sensor 630, and the gas concentration detection sensor 640 may be capable of operating concurrently in parallel. Thus, within the energy storage system 600, fire response scenarios corresponding to the respective detection sensors may be executed in parallel simultaneously. With this configuration, the battery management system (BMS) 660 can quickly and accurately detect and identify abnormally behaving battery cells, thereby facilitating early fire suppression and minimizing fire propagation.

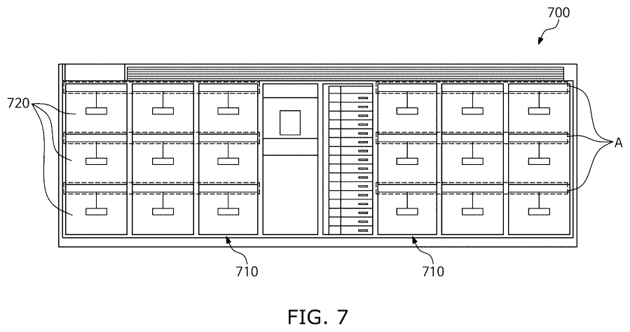

FIG. 7 is a diagram illustrating an installation location A of a branch pipe within an energy storage system 700 according to one embodiment of the present disclosure. In one embodiment, the energy storage system 700 may include a plurality of battery modules 720, and at least one battery rack 710 in which the plurality of battery modules 720 are accommodated. Within each of the battery modules 720, a plurality of battery cells may be accommodated.

Each of the battery modules 720 may have a substantially cuboid shape, and the plurality of battery modules 720 may be arranged in columns (a vertical (up and down) direction based on FIG. 7) and rows (a horizontal (left and right) direction based on FIG. 7) of the battery rack 710. Although not shown, the plurality of battery modules 720 may also be arranged in a depth direction (a front-to-back direction based on FIG. 7) of the battery rack 710. Within each of the battery modules 720, the plurality of battery cells may be arranged in a single column or multiple columns. While FIG. 7 illustrates the arrangement of three rows and three columns of the battery modules 720 within the battery rack 710, the arrangement is not limited thereto. For example, the battery modules 720 may be arranged in fewer than three rows or more than three rows and fewer than three columns or more than three columns within the battery rack 710.

As described above in FIG. 5, a main pipe included in the fire extinguishing system may extend from the agent container and may be positioned adjacent to or inserted into the plurality of battery racks 710. The main pipe may extend in a direction parallel the alignment of the plurality of battery modules 720 within the battery rack 710 (e.g., the horizontal direction based on FIG. 7). In order to facilitate the smooth transfer of the extinguishing agent through branch pipes, the main pipe may be disposed and installed above the batter rack 710 or in an upper region of the battery rack 710.

The branch pipes that branches from the main pipe and connects with the battery modules 720 may be positioned at the indicated installation locations A. Specifically, the branch pipes may be located at the upper region of the battery modules 720, allowing the branch pipes to be placed above each of the respective battery cells. The branch pipes may extend in a direction parallel to a direction in which the plurality of battery cells are aligned within each battery module 720 (e.g., a front-to-back direction based on FIG. 7). Further, the number of branch pipes connected to each battery module 720 may correspond to the number of rows of battery cells stored in the battery module 720, such that at least one branch pipe is installed for each row.

With this configuration, the branch pipes may be connected to all of the battery modules 720 included in the energy storage system 700. Further, by connecting the branch pipes to the upper region of the respective battery modules 720, the extinguishing agent can be supplied in the direction of gravity to all of the battery cells or battery cells experiencing an event, even when the plurality of battery modules 720 are aligned adjacent to each other in a front-to-back direction, a left and right direction, and/or a up and down direction.

FIG. 8 is a perspective view illustrating an example of a branch pipe 840 and an extinguishing nozzle 850 according to one embodiment of the present disclosure. In one embodiment, a main pipe 830 extending from the agent container branches into branch pipes 840, which may be connected to or disposed in an upper region of the battery module 810. Each branch pipe 840 may be disposed above the respective battery cells 820 housed within the battery module 810.

FIG. 8 shows an enlarged perspective view of a region B illustrating a bottom surface of the branch pipe 840 and the extinguishing nozzle 850. Referring to the enlarged perspective view of the region B, a plurality of extinguishing nozzles 850 may be provided in the branch pipe 840. In one embodiment, the extinguishing nozzles 850 may be respectively provided at positions above the battery cells 820 along the branch pipe 840. Specifically, the extinguishing nozzles 850 may be provided at locations corresponding to locations of vents of the battery cells 820, respectively. Here, the vent is formed at the top surface of each battery cell 820.

In one embodiment, the extinguishing nozzles 850 may be connected with passageways formed at the busbar holder (e.g., passageways 232 shown in FIG. 2). Specifically, the branch pipe 840 may be positioned above the busbar holder of the battery module 810, and the plurality of extinguishing nozzles 850 provided in the branch pipe may be respectively disposed at positions above the passageways. Therefore, the extinguishing agent sprayed through the extinguishing nozzles 850 may be directed through the passageways to the corresponding vents of the battery cells 820.

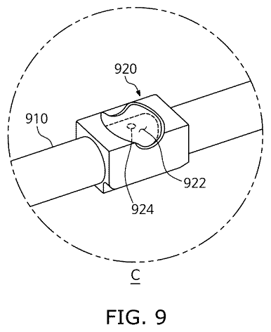

FIG. 9 is a perspective view illustrating an example of a heat-sensitive member 922 included in an extinguishing nozzle 920 according to one embodiment of the present disclosure. FIG. 9 is an enlarged perspective view of a region C of FIG. 8, showing a partial bottom surface of a branch pipe 910. The extinguishing nozzle 920 may be formed at the branch pipe 910. In one embodiment, the extinguishing nozzle 920 may include the heat-sensitive member 922. The heat-sensitive member 922 may be provided on one surface (e.g. a bottom surface) of the extinguishing nozzle 920 in a direction (e.g., gravity direction) in which the extinguishing agent is sprayed from the extinguishing nozzle 920. In FIG. 9, the extinguishing nozzle is illustrated as having a roughly cuboid shape; however, the shape is not limited thereto, and the shape of the extinguishing nozzle 920 may be appropriately modified to a polyhedral shape, a spherical shape, a hemispherical shape, or the like. Accordingly, the shape of the heat-sensitive element 922 may be suitably modified based on the shape of the extinguishing nozzle 920. The heat-sensitive member 922 may be provided at a position that faces towards the spraying direction of the extinguishing agent sprayed from the extinguishing nozzle 920.

The heat-sensitive member 922 may be configured to melt at a temperature equal to or greater than a predetermined threshold temperature. For example, the threshold temperature at which the heat-sensitive member 922 is configured to melt may be in a range from 80 degrees Celsius to 250 degrees Celsius. Accordingly, in the event of a fire in the battery cell, the heat-sensitive member 922 may be melted by heat or flame expelled through the vent, for example.

The material of the heat-sensitive member 922 may be determined by considering the temperature increase within the battery module in the event of a fire in the battery cell. The heat-sensitive member 922 may be made of a resin material such as, but not limited to, acrylonitrile butadiene styrene (ABS), polypropylene (PP), or the like.

The heat-sensitive member 922 may be formed in a shape that surrounds an injection hole 924 formed in the branch pipe 910. In this case, the heat-sensitive member 922 may normally block the injection hole 924, but in the event of a fire, the heat may melt the heat-sensitive member 922 and the injection hole 924 may be opened. When the injection hole 924 is opened, the pressure in that area is lowered, so that the extinguishing agent transferred to the branch pipe 910 can be injected and sprayed through the injection hole 924 due to the pressure gradient. The extinguishing agent may be injected and sprayed through the injection hole 924 directly onto the top of the battery cell where the fire occurs (e.g., the vent of the battery cell).

FIG. 9 illustrates that the heat-sensitive member 922 is formed in a shape to surround an area larger than the injection hole 924, but the shape of the heat-sensitive member 922 is not limited thereto. For example, the heat-sensitive member 922 may be formed in a shape corresponding to a size of the diameter of the injection hole 924. Further, the number of injection holes 924 formed in the branch pipe 910 may be two or more, and the size, location, and arrangement of the injection holes 924 may be varied as appropriate.

The heat-sensitive member 922 may be formed of a material and/or a thickness that can withstand the injection pressure of the extinguishing agent. In addition, the time during which the injection hole 924 is opened may be adjusted by adjusting the shape, material, thickness, etc. of the heat-sensitive member 922.

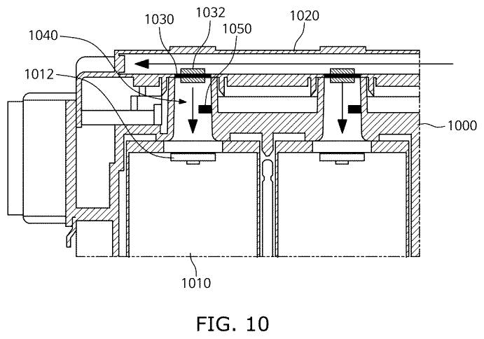

FIG. 10 is a cross-sectional view illustrating an example in which an extinguishing agent is supplied to a battery cell 1010 through an extinguishing valve 1032 according to one embodiment of the present disclosure. In a battery module 1000, a plurality of battery cells 1010 may be arranged adjacent to each other. A busbar holder may be disposed at the top of the battery module 1000, and a plurality of passageways 1040 penetrating towards the battery cells 1010 may be formed at the busbar holder. Each passageway 1040 may be formed at a position corresponding to a position of the vent 1012 formed at the top surface of the battery cell. A gas flow detection sensor 1050 may be disposed inside the passageway 1040 to detect the flow of gas discharged from the vent 1012. In another example, the gas flow detection sensor 1050 may be disposed near to the passageway 1040 on the bottom surface of the busbar holder.

A branch pipe 1020 extending from a main pipe may be located above the busbar holder. A plurality of extinguishing nozzles 1030 may be formed at the branch pipe 1020. The plurality of extinguishing nozzles 1030 may be provided at positions corresponding to positions of the passageways 1040 of the busbar holder, respectively.

Additionally, an extinguishing valve 1032 that opens and closes the extinguishing nozzle 1030 may be provided at the branch pipe 1020. The extinguishing valve 1032 may be electrically connected to the battery management system (BMS). The extinguishing valve 1032 may open and close the extinguishing nozzle 1030 in response to an instruction from the battery management system (BMS).

In one embodiment, in the event of a fire in a particular battery cell 1010, vent gas may be generated inside the battery cell 1010. The generated vent gas may be discharged through the vent 1012 formed at the top surface of the battery cell 1010. The discharged vent gas may flow through the passageway 1040 formed on the top of the battery cell 1010. In one embodiment, an outlet pipe for venting the vent gas may be disposed at the top of the busbar holder. In this case, the vent gas discharged through the vent 1012 may be discharged through the passageway 1040 to the outside along the outlet pipe.

In one embodiment, the gas flow detection sensor 1050 may be positioned above the battery cell 1010. The gas flow detection sensor 1050 may detect a flow of vent gas discharged through the vent 1012. In response to a case where the gas flow detection sensor 1050 detects the flow of gas (e.g., at least one of a wind speed, a wind pressure, or an air volume of the vent gas) that is equal to or greater than a predetermined threshold, the gas flow detection sensor 1050 may transmit a gas flow detection signal to the battery management module (BMM). In this case, the battery management module (BMM) may transmit an extinguishing preparation signal to the battery management system (BMS).

The battery management system (BMS) may control a main valve of an agent container to be opened in response to the extinguishing preparation signal. In this case, an extinguishing agent may be transferred through the main pipe and the branch pipe 1020 extending from the agent container to the top of the battery cell 1010. Accordingly, the extinguishing agent may be in a state where it can be sprayed onto the top of the battery cell 1010 as soon as the extinguishing nozzle 1030 is opened.

In a case where a fire is detected by the fire detection sensor, the fire detection sensor may transmit a fire detection signal to the battery management system (BMS). In this case, the battery management system (BMS) may control the extinguishing valve 132 associated with the battery cell 1010 where the flow of gas is detected to be opened. In response, the extinguishing agent transferred through the branch pipe 1020 may be sprayed from the extinguishing nozzle 1030. The extinguishing agent may be injected along the passageway 1040 into the upper portion (e.g., the vent) of the battery cell 1010.

In some embodiments, the main valve of the agent container (fire extinguishing vessel) can be opened in advance by detecting the flow of vent gas before a fire occurs in the battery cell 1010. Further, in the event of the fire in the battery cell 1010, the fire can be prevented from spreading to neighboring battery cells or neighboring battery modules by delivering the fire extinguishing agent directly to the battery cell 1010 (e.g., the battery cell for which the vent gas flow was detected). Furthermore, even if the fire spreads to the neighboring battery cells, the branch pipes associated with the neighboring battery cells have already been supplied with the extinguishing agent, so that the extinguishing valves can be opened to immediately supply the extinguishing agent when the gas flow detection sensors associated with the neighboring battery cells detect the flow of gas. This minimizes the damage caused by the spread of the fire.

Although the present disclosure has been described above with respect to embodiments thereof, the present disclosure is not limited thereto. Various modifications and variations can be made thereto by those skilled in the art within the spirit of the present disclosure and the equivalent scope of the appended claims.

1. A battery module comprising:
a plurality of battery cells;
a plurality of busbars electrically connecting the plurality of battery cells;
a busbar holder positioned above the plurality of battery cells and supporting the plurality of busbars;
a plurality of gas flow detection sensors fixedly coupled to the busbar holder; and
circuitry electrically connected to the plurality of busbars and to the plurality of gas flow detection sensors.

2. The battery module of claim 1, wherein the plurality of gas flow detection sensors comprises sensor bodies and a plurality of first engagement portions.

3. The battery module of claim 2, wherein the busbar holder comprises a plurality of second engagement portions corresponding to respective ones of the plurality of first engagement portions, and
the plurality of second engagement portions is positioned at a bottom surface of the busbar holder to face the plurality of battery cells.

4. The battery module as claimed in claim 3, wherein the plurality of gas flow detection sensors is fixedly coupled to the bottom surface of the busbar holder by respectively engaging the plurality of first engagement portions with the plurality of second engagement portions.

5. The battery module as claimed in claim 3, wherein a first one of the plurality of first engagement portions is formed in a protruding hook shape,
a first one of the plurality of second engagement portions is formed in a groove shape for receiving the first one of the plurality of first engagement portions, and
the first one of the plurality of first engagement portions is engaged with the first one of the plurality of second engagement portions by inserting the first one of the plurality of first engagement portions into the first one of the plurality of second engagement portions.

6. The battery module as claimed in claim 1, wherein vents are respectively formed at top surfaces of the plurality of battery cells, and
the plurality of gas flow detection sensors is respectively disposed above the vents of the plurality of battery cells.

7. The battery module as claimed in claim 6, wherein each of the plurality of gas flow detection sensors detects at least one of a wind pressure, a wind speed, or an air volume of vent gas discharging from a corresponding vent of the vents.

8. An energy storage system (ESS) comprising:
the battery module as claimed in claim 1; and
a battery rack in which the battery module is accommodated.

9. The energy storage system as claimed in claim 8, further comprising:
a fire extinguishing system; and
a battery management system (BMS) electrically connected to the battery module,
wherein the circuitry of the battery module comprises a battery management module (BMM),
each of the plurality of gas flow detection sensors transmits, in response to detecting a flow of gas, a gas flow detection signal to the BMM, and
the BMM transmits, in response to receiving the gas flow detection signal, an extinguishing preparation signal to the BMS.

10. The energy storage system as claimed in claim 9, wherein the fire extinguishing system comprises:
an agent container storing an extinguishing agent;
a main valve configured to open and close the agent container;
a main pipe through which the extinguishing agent is transferred from the agent container;
branch pipes branching from the main pipe to supply the extinguishing agent to each of the plurality of battery cells; and
a plurality of extinguishing nozzles provided along each of the branch pipes at positions corresponding to positions of the plurality of battery cells, respectively,
wherein, in response to receiving the extinguishing preparation signal, the BMS controls the main valve to be opened.

11. The energy storage system as claimed in claim 10, wherein each of the plurality of extinguishing nozzles comprises a heat-sensitive member that is configured to melt in response to a temperature of a corresponding battery cell of the plurality of battery cells being equal to or greater than a predetermined threshold, the melting of the heat-sensitive member being configured to cause the extinguishing agent to be sprayed onto the corresponding battery cell of the plurality of battery cells.

12. The energy storage system as claimed in claim 11, wherein each of the branch pipes is formed by injection holes disposed at positions respectively corresponding to battery cells of the plurality of battery cells, and
the heat-sensitive member is disposed at a position in which the heat-sensitive member blocks one of the injection holes.

13. The energy storage system as claimed in claim 10, wherein:
vents are respectively formed at top surfaces of each of the plurality of battery cells,
gas flow detection sensors of the plurality of gas flow detection sensors are respectively disposed above the vents, and
the fire extinguishing system further comprises:
a fire detection sensor; and
a plurality of extinguishing valves that open and close the plurality of extinguishing nozzles, respectively,
wherein the fire detection sensor transmits, in response to detecting a fire, a fire detection signal to the BMS, and
the BMS controls, in response to receiving the fire detection signal, a particular extinguishing valve to be opened among the plurality of extinguishing valves, the particular extinguishing valve being associated with a particular battery cell among the plurality of battery cells for which the flow of gas has been detected.

14. The energy storage system as claimed in claim 13, wherein the extinguishing agent is transferred from the agent container to the main pipe and the branch pipes after the BMS receives the extinguishing preparation signal and before the BMS receives the fire detection signal.

15. The energy storage system as claimed in claim 13, wherein, upon opening the particular extinguishing valve, the extinguishing agent is immediately sprayed into the particular battery cell through a particular extinguishing nozzle associated with the particular extinguishing valve.

16. The energy storage system as claimed in claim 13, wherein the fire detection sensor comprises a smoke detection sensor.

17. The energy storage system as claimed in claim 13, wherein, in response to receiving at least one of the extinguishing preparation signal or the fire detection signal, the BMS transmits an alert signal to an external device.

18. The energy storage system as claimed in claim 10, wherein the fire extinguishing system further comprises:
a gas concentration detection sensor; and
an active venting unit,
wherein, in response to detecting, by the gas concentration detection sensor, a gas concentration that is equal to or greater than a predetermined threshold, the gas concentration detection sensor is configured to transmit a gas detection signal to the BMS, and
the BMS is configured to activate the active venting unit in response to receiving the gas detection signal.

19. The energy storage system as claimed in claim 10, wherein the fire extinguishing system further comprises:
a gas concentration detection sensor; and
a door that opens and closes a first side of the energy storage system,
wherein, in response to detecting, by the gas concentration detection sensor, a gas concentration that is equal to or greater than a predetermined threshold, the gas concentration detection sensor is configured to transmit a gas detection signal to the BMS, and
the BMS is configured to control the door to cause the door to be opened in response to receiving the gas detection signal.

20. The energy storage system as claimed in claim 19, wherein, in response to the door being opened, the BMS is configured to transmit a door opening notification to an external device.
